SEQUENCE LISTING (17 sequences)

SEQ ID NO: 1 - DNA, 3003 bp, Artificial Sequence
  Description of Artificial Sequence Synthetic polynucleotide
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacacc

SEQ ID NO: 2 - DNA, 2995 bp, Artificial Sequence
  Description of Artificial Sequence Synthetic polynucleotide
tcctcctgctcccctcctacctctggtggaccatcgaccgcgcccggctccggtgcctgcaccgcacgccacatgcggccgatgggcagcggaagctagcagcccggcttgaatgacctatattatatgaagaagatgcctcagatgctactcgactacaaggtttatgtttctcaggtgactaaatagatcatgtgctgatgttagcgattcttgtgtggaagtgaattcatgccaatttgaagccaatatggtttaggtgtattttttactggatttaggttgaaggtcaatttgtcttctttttattctgcaaatacacattgacaacctaatgactttctattgttttaatatcagaccaataaaccttttttctttttttaatatgctatgaactgtatcagctttgtgacctcttgtttccatttccccttggattcatataattaactttcgacaccagagcaatacatctgtcatcaattattaacataatatgttatgtcttgcttggtttagcctcaagaggttttatgcatgttcttttgtattgcttggggtgttaactttttttatccatttggtgtgggtgagggtgggatctgttattcctgatgtaatgcataatccattcattatgatcatagtaggaatatggctacactgactctttcttcctcagtggagtacaattggtggccttatatacttgatgtgggcgtcatttttacgattgcctctgaatgtgggagtagtcttatggtaataaaagtcaaacaattctaatgtgttgtgctagatctaaattaccttgaagacattgttgggggttccaatgtcactgttgacatcatatccaaatttgtatccttctgtatggtgtgatatatctagaagctctaagaatttttgtcttgtgcaggtcctgtaattaatgaatgatttgaaccaaagtgttcgaggtactgttctcttgtattgaggttagttaattccatatttgatactctataggcctactttagttgacattttgattttttccacacccataatgactggtgtgatatctatttccattgatcttgttcaatttcccaatagttacatcctacatttacaacctggagagattgaagcatttttatagcaacatctgaactattaaaactcaccgtttgctccaccacgggcttaggttcttcgacctctctatgaatccccctaagataccagaactgttgtagtggttatatatattgagtatctgtttgaattgtaagaccttgtgatatttccagaagatttgtataagtctgtaatttgttgtgataatattagcatctaaatgatgcaattgatataacattattaaaatcataaatagaagtttgcatggtaccgatggttgcaatgtagtggtgaaataactatattaaaataacaaaatgtatgtatggctagctaggatttataaaatctttttcttataacacatatttgtatataaattatcatgatattatatgttcccgttgcaacgcacgggcacttatatatatatatgtgtgtgttttttttttcacatgtacccatcagataggatgataagagaggttaaatcatgccttaaggaacatcttaagaagtgtttttacatgctacattttggtggattttatataaccgttttttacatacatacggccctatatatatcatagttcagtttgattcctccgttacaaaccaactaaatgcatagaccacgcggaccgaaagcaacagggtcgatgagtcgaagcagcggggccgatgaagtcgaagcggtctcctgaacgcagatgcacgtcggcgatcgggatggctgggatggcgacgcagttgtgagtagaggcgaaaacttaatttgtgttgggattgacactaggcgccttatatagggccgtgtccacgaaccgataacgatgcgcgatccgatctacacgttatccacgaatcgatagactcgcgttccgttcatatccttatcgggatcggttagggctctaaaattaacagccaagcaacagcctcggcccggcgaggcgagcgcgtgtggttctccacactctctcctctcatccatgacttggtgagtgagcgtagcatccatatttaaactagttccactccacttgaactagcaatatgacactatttgtttcaccattctctagccataccatacatgcgcttttgagatttttttaggatttaattgaatttctcaattgggcctatcccataaatccaacacgatataagtctatctgtcgctggtagattgagagatgatgtgtgcatgtctgtaaataaaaaaaattgcttttacacataaattgcgctatgactttacatgaaataaattttctaaaatttaaaacttacataagtaaaaaaaatataaagaaggaagaaacacgacatggaaaaaaaatctctcgttgttttatatggaggcaacggctgcagtccccgtgcaagcgatgctcatccgttcccatggcgtgcacggcccagaaacgacacgcttcacctacttcttccctgccaccacacccaccgtccacccacaccacaccgcgcgccacgcgcccacggcacctcggcacagtgtcgtcgcatgtcgctcacgtactgtcgcagaactcacaccgtcacacggtgcctgctatctagctaatgctgctagcagccatgtcacaccgatataacccggccaccgcgcgccgcgccacgtcgccatgcacgcggccacgtccccgatcgatcgacgtcgtcctcctcatcctggctcctccattcccgcgcttctataaatacctcggccatgtacatcgacccagccatctcctcaccctcgttcaccacacagcccgccactcctttagtagcttgtgatttgtacgtcgacgagatcactggttggcggacgacgacccgggcaacc

SEQ ID NO: 3 - DNA, 2528 bp, Artificial Sequence
  Description of Artificial Sequence Synthetic polynucleotide
gcgccaagcttgcgctccctgacgctgtcttgggagagctgcaagatgagacactccatcccgcgcagccctgtcgtggcgtcctcctggatggacacctgcatcgctgtcgccctccaccaactcacctgaacgaagaatagaataaaaaatggagggagctgagggggcagtggttgcgctgtagggaggagagagaccgcgtcattataagactatctgcaaccgttacctctaaatttttccctctatatcattttttccccatattttcccccctattttttcatctcccgcaacggtttctcctaaatactccccctatatctcactaccactataaaatattattttttataccaactatcaattttttatctactaacaattactcgtggacccacagcacagtgtttaggagatgaacagtgacacgctatatctggggggagagagaaagaggccggcgcgtagggggcgccgtaggggcactgctgcggctgtagagtaccccctacacgccgcatgcaagggaagggggctgtcaggggggcaatgttgcgcatagcctaaagagcggatgaagcggcttgcaatttgcacgctggattcataaatagtgcatattactaaaaaaaagggtggggataggtatagagagtctattagagttgatctaagacccggtttatttcagattataatctgtccggattatataatccagcgcaaataatacagtaggtaaacaaacaactagattatgggttcagattatataatctaaaccccagattatgataatctcataatctcctcaagagtagcttattggagattattttggcaaaagacccactacccatggttatgtaaatagaaattataatatatatcatcttttttctcaccttaaataaacaaataagggtattgttgtctttatgaataatctacatttgtataatctaaactaccaaacaactacatctagattataatctggattatataatttaaattataatctagattatataatttataagctgaaacaacccggccctaaagcactatcgtatcacctatctgaaataagtcacgggtttcgaacgtccacttgcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggattgagtacaatactcatgtgcatccaaacaaactacttatattgaggtgaatttggatagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatgtataagtgttctggatattggttgttggctccatattcacacaacctaatcaatagaaaacatatgttttattaaaacaaaatttatcatatatcatatatatatatataaaccgtagcaatgcacgggcatataactagtgcaacttaatacatgtgtgtattaagatgaataagagggtatccaaataaaaaacttgttcgcttacgtctggatcgaaaggggttggaaacgattaaatctcttcctagtcaaaattgaatagaaggagatttaatctctcccaatccccttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgac

SEQ ID NO: 4 - protein, 24 aa, Hordeum sp.
MANKHLSLSLFLVLLGLSASLASG

SEQ ID NO: 5 - protein, 574 aa, Artificial Sequence
  Description of Artificial Sequence Synthetic polypeptide
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 6 - DNA, 72 bp, Hordeum sp.
atggccaacaagcacctgagcctctccctcttcctcgtgctcctcggcctctccgcctccctcgccagcggc

SEQ ID NO: 7 - DNA, 1728 bp, Artificial Sequence
  Description of Artificial Sequence Synthetic polynucleotide
gaggatgacataattattgcgacaaagaatggaaaagtcaggggtatgaatctcacggtctttggcggtacggtgaccgcgttcctgggcattccgtacgctcagccacccctaggtaggctgaggttcaaaaagcctcagtctctcacaaagtggtccgacatctggaatgccacgaagtacgctaactcctgttgccagaacattgaccagagcttccccggtttccacgggtcggaaatgtggaacccgaacaccgatctgagcgaggactgcttgtacctgaacgtttggattcccgccccaaagcccaagaacgcgaccgtcctcatctggatatacgggggtggcttccagactgggacatcctctcttcacgtatacgatggaaagttcttggctcgcgtggagcgcgtcattgtggtttccatgaactatcgggttggtgctcttggcttcctggcccttcctgggaacccggaggcaccgggcaacatgggcctctttgaccagcagttggcgctccaatgggtgcaaaagaatatcgctgctttcggcggcaaccctaaaagcgtcacactatttggcgagtccgctggagccgcatcggtgagcttgcaccttctgagccccggctcccattccctattcacaagagcaatcctccaatcaggctcctttaacgcaccttgggctgtgactagcctctacgaggcacggaaccgtacactgaacttggccaaactgactggatgcagcagggaaaatgaaactgagataattaagtgtttgagaaacaaggatccccaagagatacttcttaacgaggcctttgtggttccatacggaacccccctgtccgtaaactttggccctacagtcgatggcgatttcctcacagacatgccagatattcttctggagctcggacagtttaaaaagacccagatactggttggagtgaacaaggacgaggggactgctttcctcgtgtacggcgccccaggcttctcgaaagataacaactcaatcattacgcgcaaagagttccaggagggtctcaaaatcttctttcccggggtgtctgagtttggcaaagagtctatcctgttccattacacggactgggtggatgaccaaagaccagagaactatagggaggccttgggtgacgtggttggggattacaacttcatctgcccggctctggagttcaccaagaagttctctgagtggggaaacaacgcattcttctattacttcgagcacaggtcatcgaagctcccgtggcctgagtggatgggggtgatgcatggctatgaaattgaattcgtatttggactgcctctcgagaggcgggacaactacaccaaggccgaggagatcctgagccgctccatagtcaagcgatgggcgaacttcgcaaaatatggcaatcctaatgagacgcagaataacagcacatcatggccagtctttaagtcaaccgaacaaaaatacctgaccctcaataccgaatccacccggatcatgacaaaactccgcgcacaacagtgtcggttctggactagcttcttcccaaaggtgctggagatgactgggaacatcgacgaggcggagtgggaatggaaggcgggctttcacaggtggaataactatatgatggactggaaaaaccagttcaatgattacactagcaagaaagagagttgcgtcggtctctgataa

SEQ ID NO: 8 - DNA, 129 bp, Artificial Sequence
  Description of Artificial Sequence Synthetic polynucleotide
atggcccacgcccgcgtcctcctcctggcgctcgccgtcctggccacggccgccgtcgccgtcgcctcctcctcctccttcgccgactccaacccgatccggccggtcaccgaccgcgccgcgtccacc

SEQ ID NO: 9 - DNA, 12 bp, Artificial Sequence
  Description of Artificial Sequence Synthetic oligonucleotide
aaggacgagctg

SEQ ID NO: 10 - DNA, 18 bp, Artificial Sequence
  Description of Artificial Sequence Synthetic oligonucleotide
tccgagaaggacgagctg

SEQ ID NO: 11 - protein, 598 aa, Artificial Sequence
  Description of Artificial Sequence Synthetic polypeptide
MANKHLSLSLFLVLLGLSASLASGEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 12 - DNA, 1800 bp, Artificial Sequence
  Description of Artificial Sequence Synthetic polynucleotide
atggccaacaagcacctgagcctctccctcttcctcgtgctcctcggcctctccgcctccctcgccagcggcgaggatgacataattattgcgacaaagaatggaaaagtcaggggtatgaatctcacggtctttggcggtacggtgaccgcgttcctgggcattccgtacgctcagccacccctaggtaggctgaggttcaaaaagcctcagtctctcacaaagtggtccgacatctggaatgccacgaagtacgctaactcctgttgccagaacattgaccagagcttccccggtttccacgggtcggaaatgtggaacccgaacaccgatctgagcgaggactgcttgtacctgaacgtttggattcccgccccaaagcccaagaacgcgaccgtcctcatctggatatacgggggtggcttccagactgggacatcctctcttcacgtatacgatggaaagttcttggctcgcgtggagcgcgtcattgtggtttccatgaactatcgggttggtgctcttggcttcctggcccttcctgggaacccggaggcaccgggcaacatgggcctctttgaccagcagttggcgctccaatgggtgcaaaagaatatcgctgctttcggcggcaaccctaaaagcgtcacactatttggcgagtccgctggagccgcatcggtgagcttgcaccttctgagccccggctcccattccctattcacaagagcaatcctccaatcaggctcctttaacgcaccttgggctgtgactagcctctacgaggcacggaaccgtacactgaacttggccaaactgactggatgcagcagggaaaatgaaactgagataattaagtgtttgagaaacaaggatccccaagagatacttcttaacgaggcctttgtggttccatacggaacccccctgtccgtaaactttggccctacagtcgatggcgatttcctcacagacatgccagatattcttctggagctcggacagtttaaaaagacccagatactggttggagtgaacaaggacgaggggactgctttcctcgtgtacggcgccccaggcttctcgaaagataacaactcaatcattacgcgcaaagagttccaggagggtctcaaaatcttctttcccggggtgtctgagtttggcaaagagtctatcctgttccattacacggactgggtggatgaccaaagaccagagaactatagggaggccttgggtgacgtggttggggattacaacttcatctgcccggctctggagttcaccaagaagttctctgagtggggaaacaacgcattcttctattacttcgagcacaggtcatcgaagctcccgtggcctgagtggatgggggtgatgcatggctatgaaattgaattcgtatttggactgcctctcgagaggcgggacaactacaccaaggccgaggagatcctgagccgctccatagtcaagcgatgggcgaacttcgcaaaatatggcaatcctaatgagacgcagaataacagcacatcatggccagtctttaagtcaaccgaacaaaaatacctgaccctcaataccgaatccacccggatcatgacaaaactccgcgcacaacagtgtcggttctggactagcttcttcccaaaggtgctggagatgactgggaacatcgacgaggcggagtgggaatggaaggcgggctttcacaggtggaataactatatgatggactggaaaaaccagttcaatgattacactagcaagaaagagagttgcgtcggtctctgataa

SEQ ID NO: 13 - DNA, 1857 bp, Artificial Sequence
  Description of Artificial Sequence Synthetic polynucleotide
atggcccacgcccgcgtcctcctcctggcgctcgccgtcctggccacggccgccgtcgccgtcgcctcctcctcctccttcgccgactccaacccgatccggccggtcaccgaccgcgccgcgtccaccgaggatgacataattattgcgacaaagaatggaaaagtcaggggtatgaatctcacggtctttggcggtacggtgaccgcgttcctgggcattccgtacgctcagccacccctaggtaggctgaggttcaaaaagcctcagtctctcacaaagtggtccgacatctggaatgccacgaagtacgctaactcctgttgccagaacattgaccagagcttccccggtttccacgggtcggaaatgtggaacccgaacaccgatctgagcgaggactgcttgtacctgaacgtttggattcccgccccaaagcccaagaacgcgaccgtcctcatctggatatacgggggtggcttccagactgggacatcctctcttcacgtatacgatggaaagttcttggctcgcgtggagcgcgtcattgtggtttccatgaactatcgggttggtgctcttggcttcctggcccttcctgggaacccggaggcaccgggcaacatgggcctctttgaccagcagttggcgctccaatgggtgcaaaagaatatcgctgctttcggcggcaaccctaaaagcgtcacactatttggcgagtccgctggagccgcatcggtgagcttgcaccttctgagccccggctcccattccctattcacaagagcaatcctccaatcaggctcctttaacgcaccttgggctgtgactagcctctacgaggcacggaaccgtacactgaacttggccaaactgactggatgcagcagggaaaatgaaactgagataattaagtgtttgagaaacaaggatccccaagagatacttcttaacgaggcctttgtggttccatacggaacccccctgtccgtaaactttggccctacagtcgatggcgatttcctcacagacatgccagatattcttctggagctcggacagtttaaaaagacccagatactggttggagtgaacaaggacgaggggactgctttcctcgtgtacggcgccccaggcttctcgaaagataacaactcaatcattacgcgcaaagagttccaggagggtctcaaaatcttctttcccggggtgtctgagtttggcaaagagtctatcctgttccattacacggactgggtggatgaccaaagaccagagaactatagggaggccttgggtgacgtggttggggattacaacttcatctgcccggctctggagttcaccaagaagttctctgagtggggaaacaacgcattcttctattacttcgagcacaggtcatcgaagctcccgtggcctgagtggatgggggtgatgcatggctatgaaattgaattcgtatttggactgcctctcgagaggcgggacaactacaccaaggccgaggagatcctgagccgctccatagtcaagcgatgggcgaacttcgcaaaatatggcaatcctaatgagacgcagaataacagcacatcatggccagtctttaagtcaaccgaacaaaaatacctgaccctcaataccgaatccacccggatcatgacaaaactccgcgcacaacagtgtcggttctggactagcttcttcccaaaggtgctggagatgactgggaacatcgacgaggcggagtgggaatggaaggcgggctttcacaggtggaataactatatgatggactggaaaaaccagttcaatgattacactagcaagaaagagagttgcgtcggtctctgataa

SEQ ID NO: 14 - DNA, 1740 bp, Artificial Sequence
  Description of Artificial Sequence Synthetic polynucleotide
gaggatgacataattattgcgacaaagaatggaaaagtcaggggtatgaatctcacggtctttggcggtacggtgaccgcgttcctgggcattccgtacgctcagccacccctaggtaggctgaggttcaaaaagcctcagtctctcacaaagtggtccgacatctggaatgccacgaagtacgctaactcctgttgccagaacattgaccagagcttccccggtttccacgggtcggaaatgtggaacccgaacaccgatctgagcgaggactgcttgtacctgaacgtttggattcccgccccaaagcccaagaacgcgaccgtcctcatctggatatacgggggtggcttccagactgggacatcctctcttcacgtatacgatggaaagttcttggctcgcgtggagcgcgtcattgtggtttccatgaactatcgggttggtgctcttggcttcctggcccttcctgggaacccggaggcaccgggcaacatgggcctctttgaccagcagttggcgctccaatgggtgcaaaagaatatcgctgctttcggcggcaaccctaaaagcgtcacactatttggcgagtccgctggagccgcatcggtgagcttgcaccttctgagccccggctcccattccctattcacaagagcaatcctccaatcaggctcctttaacgcaccttgggctgtgactagcctctacgaggcacggaaccgtacactgaacttggccaaactgactggatgcagcagggaaaatgaaactgagataattaagtgtttgagaaacaaggatccccaagagatacttcttaacgaggcctttgtggttccatacggaacccccctgtccgtaaactttggccctacagtcgatggcgatttcctcacagacatgccagatattcttctggagctcggacagtttaaaaagacccagatactggttggagtgaacaaggacgaggggactgctttcctcgtgtacggcgccccaggcttctcgaaagataacaactcaatcattacgcgcaaagagttccaggagggtctcaaaatcttctttcccggggtgtctgagtttggcaaagagtctatcctgttccattacacggactgggtggatgaccaaagaccagagaactatagggaggccttgggtgacgtggttggggattacaacttcatctgcccggctctggagttcaccaagaagttctctgagtggggaaacaacgcattcttctattacttcgagcacaggtcatcgaagctcccgtggcctgagtggatgggggtgatgcatggctatgaaattgaattcgtatttggactgcctctcgagaggcgggacaactacaccaaggccgaggagatcctgagccgctccatagtcaagcgatgggcgaacttcgcaaaatatggcaatcctaatgagacgcagaataacagcacatcatggccagtctttaagtcaaccgaacaaaaatacctgaccctcaataccgaatccacccggatcatgacaaaactccgcgcacaacagtgtcggttctggactagcttcttcccaaaggtgctggagatgactgggaacatcgacgaggcggagtgggaatggaaggcgggctttcacaggtggaataactatatgatggactggaaaaaccagttcaatgattacactagcaagaaagagagttgcgtcggtctcaaggacgagctgtgataa

SEQ ID NO: 15 - DNA, 1812 bp, Artificial Sequence
  Description of Artificial Sequence Synthetic polynucleotide
atggccaacaagcacctgagcctctccctcttcctcgtgctcctcggcctctccgcctccctcgccagcggcgaggatgacataattattgcgacaaagaatggaaaagtcaggggtatgaatctcacggtctttggcggtacggtgaccgcgttcctgggcattccgtacgctcagccacccctaggtaggctgaggttcaaaaagcctcagtctctcacaaagtggtccgacatctggaatgccacgaagtacgctaactcctgttgccagaacattgaccagagcttccccggtttccacgggtcggaaatgtggaacccgaacaccgatctgagcgaggactgcttgtacctgaacgtttggattcccgccccaaagcccaagaacgcgaccgtcctcatctggatatacgggggtggcttccagactgggacatcctctcttcacgtatacgatggaaagttcttggctcgcgtggagcgcgtcattgtggtttccatgaactatcgggttggtgctcttggcttcctggcccttcctgggaacccggaggcaccgggcaacatgggcctctttgaccagcagttggcgctccaatgggtgcaaaagaatatcgctgctttcggcggcaaccctaaaagcgtcacactatttggcgagtccgctggagccgcatcggtgagcttgcaccttctgagccccggctcccattccctattcacaagagcaatcctccaatcaggctcctttaacgcaccttgggctgtgactagcctctacgaggcacggaaccgtacactgaacttggccaaactgactggatgcagcagggaaaatgaaactgagataattaagtgtttgagaaacaaggatccccaagagatacttcttaacgaggcctttgtggttccatacggaacccccctgtccgtaaactttggccctacagtcgatggcgatttcctcacagacatgccagatattcttctggagctcggacagtttaaaaagacccagatactggttggagtgaacaaggacgaggggactgctttcctcgtgtacggcgccccaggcttctcgaaagataacaactcaatcattacgcgcaaagagttccaggagggtctcaaaatcttctttcccggggtgtctgagtttggcaaagagtctatcctgttccattacacggactgggtggatgaccaaagaccagagaactatagggaggccttgggtgacgtggttggggattacaacttcatctgcccggctctggagttcaccaagaagttctctgagtggggaaacaacgcattcttctattacttcgagcacaggtcatcgaagctcccgtggcctgagtggatgggggtgatgcatggctatgaaattgaattcgtatttggactgcctctcgagaggcgggacaactacaccaaggccgaggagatcctgagccgctccatagtcaagcgatgggcgaacttcgcaaaatatggcaatcctaatgagacgcagaataacagcacatcatggccagtctttaagtcaaccgaacaaaaatacctgaccctcaataccgaatccacccggatcatgacaaaactccgcgcacaacagtgtcggttctggactagcttcttcccaaaggtgctggagatgactgggaacatcgacgaggcggagtgggaatggaaggcgggctttcacaggtggaataactatatgatggactggaaaaaccagttcaatgattacactagcaagaaagagagttgcgtcggtctcaaggacgagctgtgataa

SEQ ID NO: 16 - protein, 4 aa, Artificial Sequence
  Description of Artificial Sequence Synthetic peptide
KDEL

SEQ ID NO: 17 - protein, 6 aa, Artificial Sequence
  Description of Artificial Sequence Synthetic peptide
SEKDEL

(19) United States
(12) Patent Application Publication
(10) Pub. No.: US 2017/0096676 A1
(43) Pub. Date: Apr. 6, 2017

(54) Expression of Butyrylcholinesterase in plants

(71) Applicant: Applied Biotechnology Institute, Inc., San Luis Obsipo, CA (US)

(72) Inventors: Erin Egelkrout, San Luis Obispo, CA (US); John Howard, Cayucos, CA (US); Melinda E. Wales, Bryan, TX (US); Celine Hayden, San Luis Obispo, CA (US)

(21) Appl. No.: 15/194,740

(22) Filed: Jun. 28, 2016

Related U.S. Application Data
(60) Provisional application No. 62/188,850, filed on Jul. 6, 2015.

(51) Int. Cl. C12N 15/82

(52) U.S. Cl. CPC C12N 15/8257; C12N 15/8234; C12Y 301/01008; C12N 15/8241; C12N 15/8222

(57) ABSTRACT

Method and plants expressing increased levels of Butyrylcholinesterase (BChE) is described. The nucleic acid molecule encoding BChE is operably linked to a promoter preferentially expressing to the endosperm cells of the plant, another embodiment expression is targeted to the endoplasmic reticulum of plant cell(s), to the cell wall of the plant cell(s) or both.

### REFERENCE TO RELATED APPLICATION

This application claims priority to previously filed and co-pending provisional application U.S. Ser. No. 62/188,850, filed Jul. 6, 2015, the contents of which are incorporated herein by reference in its entirety.

### STATEMENT REGARDING FEDERALLY SPONSORED RESEARCH OR DEVELOPMENT

This invention was made with Government support under contract (HDTRA1-12-C-0052) awarded by U.S. Department of Defense. The Government has certain rights in the invention.

### SEQUENCE LISTING

The instant application contains a Sequence Listing which has been submitted electronically in ASCII format and is hereby incorporated by reference in its entirety. Said ASCII copy, created on Jun. 27, 2016, is named AB00017_SL.txt and is 36,460 bytes in size.

### BACKGROUND

Butyrylcholinesterase (BChE) has generated interest as a biopharmaceutical based on its ability to bind and sequester organophosphorus (OP) compounds (1). OP compounds target cholinesterase enzymes that regulate nerve transmission and were developed for use as pesticides and chemical weapons. The potential for use of chemical weapons as threat agents, as well as accidental exposure to pesticides, has led to extensive research on therapeutic countermeasures. Current treatments, such as atropine and oximes, may mitigate symptoms but not prevent long-term disability. BChE has been identified as a lead candidate for development as a bioscavenger that may be used in a more effective response to exposure to chemical weapons agents or pesticides. It may also have use in the treatment of overdoses of drugs such as cocaine.

BChE is a serine hydrolase that breaks down butyrylcholine. Although the exact physiological function of native BChE is not clear, it may have a partially redundant function with acetylcholinesterase in regulating neurotransmitter stability (2-4). In human plasma, BChE is typically found as a 340 kDa tetrameric glycoprotein. The mature tetrameric form is stabilized through interactions at the C-terminal tetramerization domains with proline rich attachment domain (PRAD) or proline rich membrane anchor (PRiMA) proteins (5-8) that anchor BChE to the cell membranes. Denaturation of BChE results in release of a number of families of different endogenous polyproline peptides, thought to be cleavage products of the same protein (9). In a more detailed study using equine BChE, just one of these was consistent with a previously reported lamellipodin proline rich peptide, suggesting that other endogenous polyproline peptides may be involved (10).

The role of BChE as a bioscavenger depends on its ability to bind OP compounds, thus preventing harm by sequestering them away from the native enzymes that regulate nerve transmission. This mechanism requires BChE in stoichiometric amounts, although significant research has been conducted to develop catalytic variants (4). It also requires that BChE be present in the bloodstream for an adequate amount of time. The tetrameric form of BChE has a relatively slow clearance rate, with a half-life of 11-14 days, and so has a more favorable pharmacokinetic profile than its monomeric counterpart. In some studies, the half-life of the tetramer on injection was found to be 16-56 hours in comparison to 2-300 minutes for the monomer (11-14). Effective use of BChE will depend on preferential production of the tetrameric form. There is a need for reliable reduced cost production of BChE in proper form, preferably in tetrameric form.

### SUMMARY

A method is shown that results in increased expression of Butyrylcholinesterase (BChE) in a plant. A nucleic acid molecule encoding BChE is operably linked to a promoter preferentially expressing BChE to endosperm cells of the plant. The method in an embodiment further provides for a nucleic acid molecule that targets expression of BChE to the endoplasmic reticulum of the plant cells and in a further embodiment to the cell wall of the plant cells, and in still another embodiment, provides for nucleic acid molecules targeting to the cell wall and the endosplasmic reticulum of the plant cells. The method results in plants expressing increased levels of BChE. Plants expressing increased levels of BChE are provided.

### BRIEF DESCRIPTION OF THE DRAWINGS

FIG. 1 is a graphic representation of the components of the constructs listed. The reference to pr25 refers to the embryo preferred promoter and pr39 to the endosperm preferred promoter described below. tBChE is the truncated butyryl cholinesterase (monomeric form). BAASS refers to the barley alpha amylase sequence described below, PinII is the terminator sequence and Vac refers to the vacuole targeting sequences described below. SEKDEL (SEQ ID NO: 17) is an endoplasmic reticulum sequence. Reference to hu28aa in the figure is to a synthesized 17 aa proline-rich peptide derived from a.a. 686-702 of human lamellipodin. The figure discloses “KDEL” as SEQ ID NO: 16.

FIG. 2A is a graph showing expression of BChE as total soluble protein expressed using constructs BSE (targeted to the cell wall) BSK (targeted to the endoplasmic reticulum) and BSJ (targeted to the vacuole). FIG. 2B shows the percent total soluble protein of all seeds produced and FIG. 2C the percent total soluble protein of the top ten highest expressing seeds.

FIG. 3 is a graph of analysis of oligomerization in BSE and BSK, showing relative activity, expression levels as mL and molecular weight of protein produced in plants using the named constructs.

FIG. 4 is a graphic representation of two constructs, BSM and BSN. KDEL (SEQ ID NO: 16) retains the expression in the endoplasmic reticulum, PinII refers to the PinII termination signal and pr39 refers to the pr39 promoter. ColQ refers to rat ColQ described below.

### DESCRIPTION

The following describes a production system that can produce large amounts of the enzyme at increased levels in plants. Currently BuChE is purified from outdated blood supplies. This route has limited utility due to its high cost (˜$20,000 per 400 mg dose (15)) and its low supply availability (16). Extraction of BuChE from plasma to produce 1 kg of enzyme has been estimated to require the entire US blood supply and would yield only a small stockpile of 2,500 doses (17). Several efforts have been made to develop a commercially viable transgenic production system for BChE, including expression in various cell lines and in transgenic goats (18). Both stable expression and transient expression using the MagniCON system have been reported in Nicotiana (17, 19-22). In CHO cells, addition of an AChE-associated collagen tail protein (ColQ) polyproline peptide allowed increased formation of tetramers (14). In the milk of transgenic mice and goats the dimeric form was predominantly produced (18). However, there are reports of lactation problems in the transgenic goats (23). In tobacco a high proportion of the tetrameric form has been produced (19). This protein was also shown to provide protection to animals on exposure to OP compounds (17).

The formation of tetramers in stably transformed tobacco suggests that endogenous polyproline peptides favors tetramer formation. Subcellular localization may also affect, or be affected by, oligomerization status of recombinant expressed BChE (22). Some of the more recent approaches in tobacco incorporate co-expression of a polyproline peptide to optimize tetramer formation (24). The tobacco work demonstrates that a plant-produced BChE is a possible means of production. In order to make this a practical approach, there must also be a means of a cost-effective purification of BChE away from endogenous toxic compounds and proteases that may impact protein stability. In addition, while transient expression systems such as MagniCON allow rapid changes to the expressed construct, the need to continually maintain Agrobacterium cultures and infiltrate plants makes this a relatively costly and labor-intensive approach. This approach also requires either indoor growth or faces significant regulatory issues with using Agrobacterium in the field.

While the tobacco system clearly shows proof-of-concept, there is a strong case for the potential use of another plant system having more favorable economics. This system would ideally be free of proteases and inherent toxic compounds and be able to express BChE without the complications of tobacco. Our goal is to develop a cost-effective system for BChE production that can be easily scaled-up and that allows for a practical method for stockpiling the enzyme in a stable form. This may then lead to a method to treat selected risk groups and a ready supply of large amounts of BChE adequate for mass populations in an emergency.

Employing plants as a means of production offers many attractive features such as eukaryotic downstream cellular processing and an animal-free source for the active ingredient. There is a wide variation in the type of plants that can be used and there are specific advantages attributable the different plant systems that can vary dramatically depending on the plant type and the end use (25, 26). One of the most promising systems has been the production of recombinant proteins in maize grain. The advantages of maize include:
1. Maize grain provides a source of protein at one of the lowest costs known (27, 28).
2. To increase the accumulation of proteins in recombinant host tissue, we have developed proprietary seed-preferred promoters and expression cassettes that have successfully raised recombinant protein expression levels to some of the highest reported for any proteins in plants (29-31), leading to a low cost of recombinant production
3. As a tissue adapted to long-term survival in a desiccated state, maize seed has high levels of endogenous protease inhibitors, which allows high stability of recombinant proteins in the host at ambient temperatures for years. This in turn allows stockpiling of active ingredient with just-in-time processing and purification.
4. Maize has the FDA's generally regarded as safe (GRAS) status that allows for reduced risk in commercialization of recombinant proteins.
5. Several recombinant protein products are currently being marketed that have been produced in maize grain providing experience in scale-up and regulatory compliance (29, 32, 33).

Using this system, we have expressed human BChE (hu-BChE) in transgenic maize. The sequence of hu-BChE was optimized for maize codon usage and expression was targeted to several subcellular locations. Increased levels of BChE can be expressed in plants, and in an embodiment in maize, using a promoter that preferentially expresses to the endosperm of plant seed. Further embodiments provide for the endosperm promoter and the nucleic acid molecule encoding BChE to be operably linked to a nucleic acid molecule targeting expression to the cell wall, targeting to the endoplasmic reticulum, or both.

The term plant composition refers to plant or plant material or plant part or plant tissue or plant cell including collection of plant cells. It is used broadly herein to include any plant at any stage of development, or to part of a plant, including a plant cutting, a plant cell culture, a plant organ, a plant seed, and a plantlet. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

When using the germ (embryo) of the plant, one can separate the germ from the remainder of the seed and use it as a source of the BChE. Such promoters are discussed below, and methods of using germ as the source of protein are discussed at U.S. Pat. Nos. 7,179,961 and 6,504,085 incorporated herein by reference in their entirety. Here, it is found that expressing preferentially to the endosperm results in increased expression of the preferred tetramer form of BChE.

A “construct” is a package of genetic material inserted into the genome of a cell via various techniques. A “vector” is any means for the transfer of a nucleic acid into a host cell. A vector may be a replicon to which a DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A cell has been “transfected” by exogenous or heterologous DNA or RNA when such DNA or RNA has been introduced inside the cell.

When referring to a nucleic acid molecule encoding BChE, is intended to include by way of example, a nucleic acid molecule that encodes the BChE protein and variants and fragments thereof. Such protein will retain its ability to bind and sequester organophosphorus (OP) compounds.

As used herein, the terms nucleic acid or polynucleotide refer to deoxyribonucleotides or ribonucleotides and polymers thereof in either single- or double-stranded form. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, and mRNA encoded by a gene.

As used herein, a nucleotide segment is referred to as operably linked when it is placed into a functional relationship with another nucleic acid segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Operably linked elements may be contiguous or non-contiguous. When used to refer to the joining of two protein coding regions, by operably linked it is intended that the coding regions are in the same reading frame. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions.

Nucleic acids include those that encode an entire polypeptide or fragment thereof. The invention includes not only the exemplified nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 86%, 87%, 88%, 89% still more preferably at least 90%, 91%, 92%, 93%, 94%, and even more preferably at least about 95%, 96%, 97%, 98%, 99%, 100% identical (or any percentage in between) to an exemplified nucleotide sequence. The nucleotide sequence may be modified as described previously, so long any antigenic polypeptide encoded is capable of inducing the generation of a protective response.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, 1984, Proteins).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques.

A protein is comprised of an amino acid sequence when the amino acid sequence is at least part of the final amino acid sequence of the protein. In such a fashion, the protein may be an original polypeptide, a variant polypeptide and/or have additional amino acid molecules, such as amino acid residues (contiguous encoded sequence) that are naturally associated with it or heterologous amino acid residues/peptide sequences. Such a protein can have a few additional amino acid residues or can comprise several hundred or more additional amino acids.

The variant proteins used in the present invention can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., eds. (1995) Current Protocols in Molecular Biology (Greene Publishing and Wiley-Interscience, New York). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Fragments of the variant proteins may be used, in addition to proteins and peptides that comprise and consist of such fragments, provided that such fragments act as an antigen and/or provide treatment for and/or protection against infections as provided by the present invention.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1.0 M NaCl, 1% SDS at 37° C., and a wash in 0.1×SSC at 60 to 65° C.

Specificity is also the function of post-hybridization washes, the critical factors being the ionic strength and temperature of the final wash solution. For DNA-DNA hybrids, the Tₘ can be approximated from the equation Tₘ=81.5° C.+16.6 (log M)+0.41(% GC)−0.61(% form.)−500/L, where M is the molarity of monovalent cations, % GC is the percentage of guanosine and cytosine nucleotides in the DNA, % form is the percentage of formamide in the hybridization solution, and L is the length of the hybrid in base pairs (Meinkoth and Wahl, 1984). The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted for sequences of the desired identity to hybridize. For example, if sequences with 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11 to 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Ausubel et al., eds. (1995) Current Protocols in Molecular Biology (Greene Publishing and Wiley-Interscience, New York) and Sambrook et al., (1989) Molecular Cloning: A Laboratory Manual, 2nd Edition. Cold Spring Harbor Laboratory Press, Plainview, N.Y.

The following terms are used to describe the sequence relationships between two or more nucleic acids or polynucleotides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length promoter sequence, or the complete promoter sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to accurately reflect the similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Optimal alignment of sequences for comparison can use any means to analyze sequence identity (homology) known in the art, e.g., by the progressive alignment method of termed “PILEUP” (Morrison, Mol. Biol. Evol. 14:428-441 (1997), as an example of the use of PILEUP); by the local homology algorithm of Smith & Waterman (Adv. Appl. Math. 2: 482 (1981)); by the homology alignment algorithm of Needleman & Wunsch (J. Mol. Biol. 48:443 (1970)); by the search for similarity method of Pearson (Proc. Natl. Acad. Sci. USA 85: 2444 (1988)); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins, Gene 73: 237-244 (1988); Corpet, Nucleic Acids Res. 16:10881-10890 (1988); Huang, Computer Applications in the Biosciences 8:155-165 (1992); and Pearson, Methods in Mol. Biol. 24:307-331 (1994); Pfam (Sonnhammer, Nucleic Acids Res. 26:322-325 (1998); TreeAlign (Hein, Methods Mol. Biol. 25:349-364 (1994); MEG-ALIGN, and SAM sequence alignment computer programs; or, by manual visual inspection.

Another example of algorithm that is suitable for determining sequence similarity is the BLAST algorithm, which is described in Altschul et al, J. Mol. Biol. 215: 403-410 (1990). The BLAST programs (Basic Local Alignment Search Tool) of Altschul, S. F., et al., (1993) J. Mol. Biol. 215:403-410) searches under default parameters for identity to sequences contained in the BLAST “GENEMBL” database. A sequence can be analyzed for identity to all publicly available DNA sequences contained in the GENEMBL database using the BLASTN algorithm under the default parameters.

Software for performing BLAST analyses is publicly available through the National Center for Biotechnology Information, www.ncbi.nlm.nih.gov/; see also Zhang, Genome Res. 7:649-656 (1997) for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al, J. Mol. Biol. 215: 403-410 (1990)). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff, Proc. Natl. Acad. Sci. USA 89:10915-10919 (1992)) alignments (B) of 50, expectation (E) of 10, M=5, N=−4, and a comparison of both strands. The term BLAST refers to the BLAST algorithm which performs a statistical analysis of the similarity between two sequences; see, e.g., Karlin, Proc. Natl. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In an embodiment, GAP (Global Alignment Program) can be used. GAP uses the algorithm of Needleman and Wunsch J. Mol. Biol. 48:443-453 (1970) to find the alignment of two complete sequences that maximizes the number of matches and minimizes the number of gaps. Default gap creation penalty values and gap extension penalty values in the commonly used Version 10 of the Wisconsin Package® (Accelrys, Inc., San Diego, Calif.) for protein sequences are 8 and 2, respectively. For nucleotide sequences the default gap creation penalty is 50 while the default gap extension penalty is 3. Percent Similarity is the percent of the symbols that are similar. Symbols that are across from gaps are ignored. A similarity is scored when the scoring matrix value for a pair of symbols is greater than or equal to 0.50, the similarity threshold. A general purpose scoring system is the BLOSUM62 matrix (Henikoff and Henikoff, Proteins, 17: 49-61 (1993)), which is currently the default choice for BLAST programs. BLOSUM62 uses a combination of three matrices to cover all contingencies. Altschul, J. Mol. Biol. 36: 290-300 (1993), herein incorporated by reference in its entirety and is the scoring matrix used in Version 10 of the Wisconsin Package® (Accelrys, Inc., San Diego, Calif.) (see Henikoff & Henikoff (1989) Proc. Natl. Acad. Sci. USA 89:10915).

As used herein, “sequence identity” or “identity” in the context of two nucleic acid sequences makes reference to the residues in the two sequences that are the same when aligned for maximum correspondence over a specified comparison window.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Identity to a sequence used herein would mean a polynucleotide sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

A nucleic acid molecule may be combined with any number of other components to be introduced into the plant, including combined with another nucleic acid molecule of interest to be expressed in the host. The “nucleic acid molecule of interest” refers to a nucleotide sequence that encodes for another desired polypeptide or protein but also may refer to nucleotide sequences that do not constitute an entire gene, and which do not necessarily encode a polypeptide or protein. For example, when used in a homologous recombination process, the nucleic acid molecule may be placed in a construct with a sequence that targets and area of the chromosome in the plant but may not encode a protein. The gene can be used to drive mRNA that can be used for a silencing system, such as antisense, and in that instance, no protein is produced. Means of increasing or inhibiting a protein are well known to one skilled in the art and, by way of example, may include, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering and, homologous recombination. In the case of use with homologous recombination, no in vivo construct will be required. If desired, a nucleic acid molecule of interest can be optimized for host or other plant translation by optimizing the codons used for host or plants and the sequence around the translational start site for host or plants. Sequences resulting in potential mRNA instability can also be avoided.

In general, the methods available for construction of recombinant genes, optionally comprising various modifications for improved expression, can differ in detail and any of the methods available to one skilled in the art may be used in the invention. However, conventionally employed methods include PCR amplification, or the designing and synthesis of overlapping, complementary synthetic oligonucleotides, which are annealed and ligated together to yield a gene with convenient restriction sites for cloning, or subcloning from another already cloned source, or cloning from a library. The methods involved are standard methods for a molecular biologist (Sambrook et al., (1989) Molecular Cloning: A Laboratory Manual, 2nd Edition. Cold Spring Harbor Laboratory Press, Plainview, N.Y.).

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to an embryo preferred promoter is meant that it expresses an operably linked sequence to a higher degree in embryo tissue that in other plant tissue. It may express during embryo development, along with expression at other stages, may express strongly during embryo development and to a much lesser degree at other times.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

In one embodiment, the expression vector also contains a gene encoding a selectable or scoreable marker that is operably or functionally linked to a promoter that controls transcription initiation. Examples of selectable markers include those that confer resistance to antimetabolites such as herbicides or antibiotics, for example, dihydrofolate reductase, which confers resistance to methotrexate (Reiss, (1994) Plant Physiol. (Life Sci. Adv.) 13:143-149; see also Herrera Estrella et al., (1983) Nature 303:209-213; Meijer et al., (1991) Plant Mol. Biol. 16:807-820); neomycin phosphotransferase, which confers resistance to the aminoglycosides neomycin, kanamycin and paromycin (Herrera-Estrella, (1983) EMBO J. 2:987-995, and Fraley et al. (1983) Proc. Natl. Acad. Sci USA 80:4803) and hygro, which confers resistance to hygromycin (Marsh, (1984) Gene 32:481-485; see also Waldron et al., (1985) Plant Mol. Biol. 5:103-108; Zhijian et al., (1995) Plant Science 108:219-227); trpB, which allows cells to utilize indole in place of tryptophan; hisD, which allows cells to utilize histinol in place of histidine (Hartman, (1988) Proc. Natl. Acad. Sci., USA 85:8047); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, (1987), in: Current Communications in Molecular Biology, Cold Spring Harbor Laboratory ed.); and deaminase from Aspergillus terreus, which confers resistance to Blasticidin S (Tamura, (1995) Biosci. Biotechnol. Biochem. 59:2336-2338). Additional selectable markers include, for example, a mutant EPSPV-synthase, which confers glyphosate resistance (Hinchee et al., (1998) BioTechnology 91:915-922), a mutant acetolactate synthase, which confers imidazolinone or sulfonylurea resistance (Lee et al., (1988) EMBO J. 7:1241-1248), a mutant psbA, which confers resistance to atrazine (Smeda et al., (1993) Plant Physiol. 103:911-917), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., (1983) EMBO J. 2:987-992); streptomycin (Jones et al., (1987) Mol. Gen. Genet. 210:86-91); spectinomycin (Bretagne-Sagnard et al., (1996) Transgenic Res. 5:131-137); bleomycin (Hille et al., (1990) Plant Mol. Biol. 7:171-176); sulfonamide (Guerineau et al., (1990) Plant Mol. Biol. 15:127-136); bromoxynil (Stalker et al., (1988) Science (1986) 242:419-423); glyphosate (Shaw et al., Science 233:478-481); phosphinothricin (DeBlock et al., (1987) EMBO J. 6:2513-2518), and the like. One option for use of a selective gene is a glufosinate-resistance encoding DNA and in one embodiment can be the phosphinothricin acetyl transferase (PAT), maize optimized PAT gene or bar gene under the control of the CaMV 35S or ubiquitin promoters. The genes confer resistance to bialaphos. See, Gordon-Kamm et al., (1990) Plant Cell 2:603; Uchimiya et al., (1993) BioTechnology 11:835; White et al., Nucl. Acids Res. 18:1062, (1990); Spencer et al., 1990) Theor. Appl. Genet. 79:625-631, and Anzai et al., (1989) Mol. Gen. Gen. 219:492. A version of the PAT gene is the maize optimized PAT gene, described at U.S. Pat. No. 6,096,947.

In addition, markers that facilitate identification of a cell containing the polynucleotide encoding the marker may be employed. Scorable or screenable markers are useful, where presence of the sequence produces a measurable product and can produce the product without destruction of the cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. (1987) The EMBO Journal vol. 6 No. 13 pp. 3901-3907); alkaline phosphatase. Other screenable markers include the anthocyanin/flavonoid genes in general (See discussion at Taylor and Briggs, (1990) The Plant Cell 2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., (1996) Plant Cell 8: 1171-1179; Scheffler et al. (1994) Mol. Gen. Genet. 242:40-48) and maize C2 (Wienand et al., (1986) Mol. Gen. Genet. 203:202-207); the B gene (Chandler et al., (1989) Plant Cell 1:1175-1183), the p1 gene (Grotewold et al, (1991 Proc. Natl. Acad. Sci USA) 88:4587-4591; Grotewold et al., (1994) Cell 76:543-553; Sidorenko et al., (1999) Plant Mol. Biol. 39:11-19); the bronze locus genes (Ralston et al., (1988) Genetics 119:185-197; Nash et al., (1990) Plant Cell 2(11): 1039-1049), among others. Yet further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PhiYFP™ from Evrogen; see Bolte et al. (2004) J Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., (1995) Plant J. 8(5):777-84); and DsRed where cells transformed with the marker gene are red in color, and thus visually selectable (Dietrich et al. (2002) Biotechniques 2(2):286-293). Additional examples include a p-lactamase gene (Sutcliffe, (1978) Proc. Nat'l. Acad. Sci. U.S.A. 75:3737), which encodes an enzyme for which various chromogenic substrates are known (e.g., PADAC, a chromogenic cephalosporin); a xylE gene (Zukowsky et al., (1983) Proc. Nat'l. Acad. Sci. U.S.A. 80:1101), which encodes a catechol dioxygenase that can convert chromogenic catechols; an α-amylase gene (Ikuta et al., (1990) Biotech. 8:241); and a tyrosinase gene (Katz et al., (1983) J. Gen. Microbiol. 129:2703), which encodes an enzyme capable of oxidizing tyrosine to DOPA and dopaquinone, which in turn condenses to form the easily detectable compound melanin. Clearly, many such markers are available to one skilled in the art.

Leader sequences can be included to enhance translation. Various available leader sequences may be substituted or added. Translation leaders are known in the art and include, for example: picornavirus leaders, for example, EMCV leader (encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165 (2):233-8); human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie. (1987) Nucleic Acids Res. 15(8):3257-73); and maize chlorotic mottle virus leader (MCMV) (Lommel et al. (1991) Virology 81:382-385). See also, Della-Cioppa et al. (1987) Plant Physiology 84:965-968.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL (SEQ ID NO: 16). This sequence (Lys-Asp-Glu-Leu) (SEQ ID NO: 16) contains the binding site for a receptor in the endoplasmic reticulum. (Munro et al., (1987) “A C-terminal signal prevents secretion of luminal ER proteins.” Cell. 48:899-907. Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

In addition to a promoter, the expression cassette can include one or more enhancers. By “enhancer” is intended a cis-acting sequence that increases the utilization of a promoter. Such enhancers can be native to a gene or from a heterologous gene. Further, it is recognized that some promoters can contain one or more enhancers or enhancer-like elements. An example of one such enhancer is the 35S enhancer, which can be a single enhancer, or duplicated. See for example, McPherson et al, U.S. Pat. No. 5,322,938. Other methods known to enhance translation can also be utilized, for example, introns, and the like. Other modifications can improve expression, include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The termination region can be native with the promoter nucleotide sequence can be native with the DNA sequence of interest, or can be derived from another source. Convenient termination regions are available from the Ti-plasmid of A. tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp7831-7846 (nos)). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions, such as transitions and transversions, can be involved.

The transformation vector comprising the sequence operably linked to a heterologous nucleotide sequence in an expression cassette, can also contain at least one additional nucleotide sequence for a gene to be cotransformed into the organism. Alternatively, the additional sequence(s) can be provided on another transformation vector.

The method of transformation/transfection is not critical; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Mild and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993) Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hi II maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the U.S. Pat. No. 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the U.S. Pat. No. 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase, and are harvested at a density of no more than OD₆₀₀=0.5, preferably between 0.2 and 0.5. The bacteria are then centrifuged to remove the media and resuspended in the co-culture medium. Since Hi II is used, medium preferred for Hi II is used. This medium is described in considerable detail by Armstrong and Green (1985). The resuspension medium is the same as that described above. All further Hi II media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity, and becomes “reprogrammed” to have a new identity. Thus the scutellum cells become embryogenic callus.

A transgenic plant may be produced that contains an introduced nucleic acid molecule encoding the BChE.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn., 4^{th} Edit.). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Any plant species may be used, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

### Examples

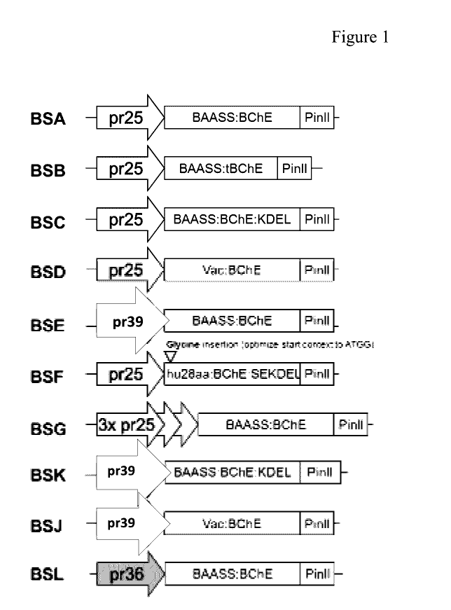

FIG. 1 shows the constructs summarized. The pr25 promoter (SEQ ID NO: 1) and pr36 promoter (SEQ ID NO: 2) are promoters preferentially expressing to the embryo of the plant cell. They are described at Streatfield S J, Bray J, Love R T, Horn M E, Lane J R, Drees C F, Egelkrout E M and Howard J A. (2010). Identification of maize embryo-preferred promoters suitable for high-level heterologous protein production. GM Crops, 1(3): 162-172. The pr39 promoter (SEQ ID NO: 3) is an endosperm preferred promoter. The promoter is discussed at Das, O. P., Poliak, E., Ward, K. and Messing, J. (1991). A new allele of the duplicated 27 kD zein locus of maize generated by homologous recombination. Nucleic Acids Res. 19 (12), 3325-3330. The reference to tBChE in BSB refers to the truncated butyrul cholinesterase (monomeric form). BAASS refers to the barley alpha amylase sequence, PinII is the terminator sequence. The term hu28aa as referred to in BSF refers to a synthesized 17 aa proline-rich peptide derived from a.a. 686-702 of human lamellipodin

Maize-optimized human BChE coding sequences were commercially synthesized (Blue Heron) with the addition of subcellular localization sequences for targeting to the cell wall, endoplasmic reticulum (ER) or vacuole. It is to be understood the targeting sequences disclosed here are for exemplification only and are not intended to limit the scope of sequences or methods of targeting. The BSE and BSL constructed used a BAASS cell wall targeting sequence and BChE sequence. The BAASS amino acid sequence encoded is SEQ ID NO: 4, and the BChE amino acid sequence is SEQ ID NO: 5. The BAASS coding sequence used is SEQ ID NO: 6 and the BChE coding region is SEQ ID NO: 7. The entire full length sequence of the BSE construct amino acid sequence is SEQ ID NO: 11 and the BSE/BSL entire nucleotide sequences is SEQ ID NO: 12. The vacuole targeting signal used in BSD and BSJ is SEQ ID NO: 8. The full length nucleotide sequence of BSJ is SEQ ID NO: 13. In BSK the construct was prepared with a BAASS signal sequence (SEQ ID NO: 6) before the BChE coding region (SEQ ID NO: 7) to aid in expression, and after the BChE coding sequence, was placed a KDEL endoplasmic reticulum targeting sequence (SEQ ID NO: 9; “KDEL” disclosed as SEQ ID NO: 16). As indicated, certain constructs used the SEKDEL endoplasmic reticulum retention sequences (SEQ ID NO: 10 “SEKDEL” disclosed as SEQ ID NO: 17). The full length sequence of the BSK construct is SEQ ID NO: 15. The synthesized coding sequence was transferred into pSB1/pSB11 vector system using the restriction enzymes PacI+NcoI or Age1+NcoI to exchange fragments with existing constructs containing the relevant promoter sequence. The constructs also contained a maize-optimized phosphinothricin acetyl transferase gene conferring resistance to the herbicide bialaphos. The constructs were transferred into Agrobacterium strain LBA4404 by standard triparental mating procedures and the resulting cointegrate was introduced into Agrobacterium strain EHA101 by electroporation.

Maize transformation was carried out using a method modified from Ishida, et al. (Ishida, et al. (1996) Nature Biotechnol. 14: 745-50). Hill Maize embryos at roughly 2-4 mm were mixed with A. tumefaciens EHA101 with the appropriate vector for transformation. Typically, 3000-5000 embryos were used for each construct for a target of 10-20 independent transformation events. The herbicide bialaphos was added to the media at 1.6 μg/mL to select for transformants. Plants from events selected on bialaphos were grown to maturity in the greenhouse and pollinated with Hill to produce T₁ seed. For production of T₂ seed T₁ seed were grown and plants were pollinated with line MS0168, an elite inbred from Stine Seed (Adel, Iowa).

Expression was determined using a modified version of the assay described by Ellman (Ellman, G. L., Courtney, K. D. & Featherstone, R. M. (1961) “A new and rapid colorimetric determination of acetylcholinesterase activity” Biochemical pharmacology. 7: 88-95). BChE activity in the extracts is evaluated in a 200 μL reaction mixture containing 20 μL of non-diluted extracts and 20 μL of 10 mM DTNB, 50 μL 7 mM S-butyrylthiocholine in 100 mM Phosphate buffer, pH 7.4. The reaction is conducted at room temperature and monitored at 415 nm. For the T₁ seed analysis the seed is pulverized and a crude extract used in 50 mM Tris, 150 mM NaCl, 0.1 mM EDTA pH 7.4 buffer.

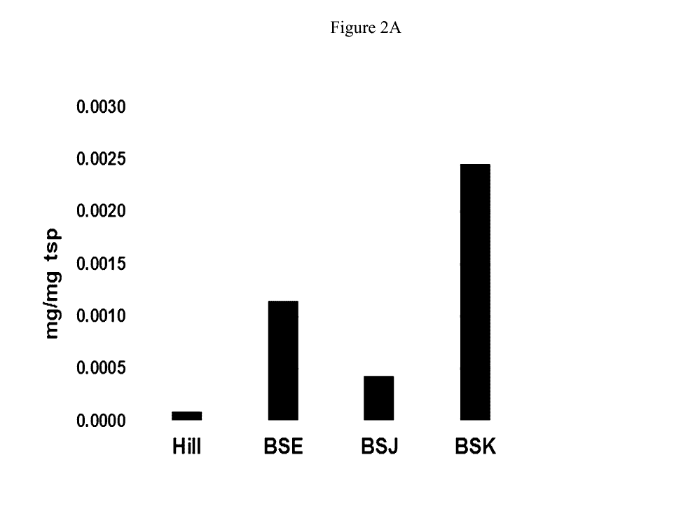

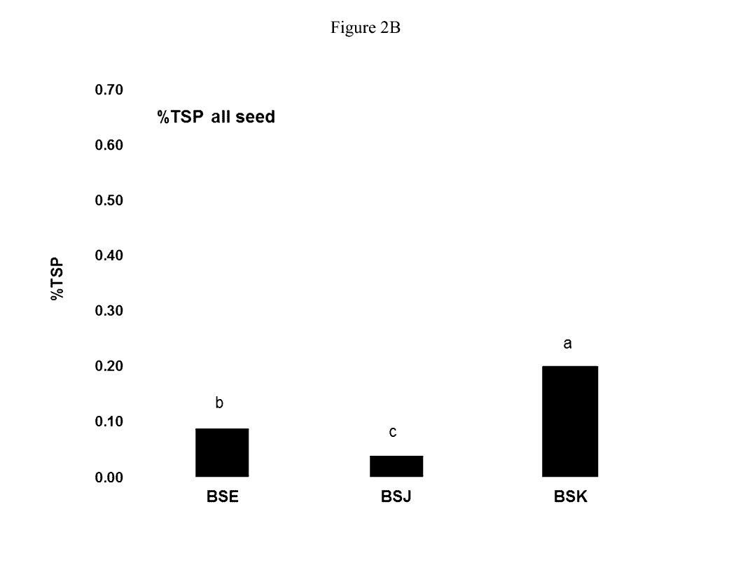

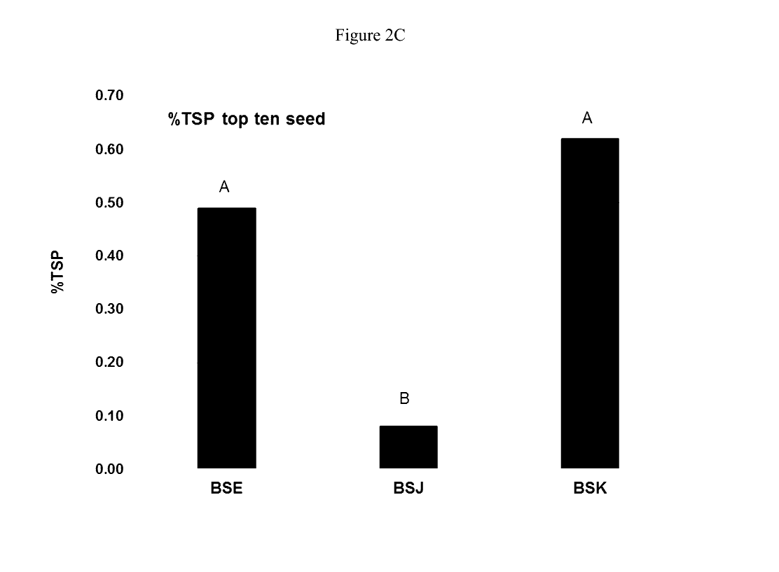

These studies have identified combinations of promoter and subcellular localization that allow high levels of expression of butyrylcholinesterase. We created seven different constructs and subsequently identified three constructs that express significant levels of BChE. Using commercially available standards, the average expression in mg BChE/mg total soluble protein for all events tested thus far is shown in FIG. 2A. This method is a useful comparison to identify the best construct but within a specific construct, individual transformation events vary dramatically. Constructs for expression of BChE under control of an endosperm-preferred promoter and targeted to the cell wall (BSE), ER (BSK), or the vacuole (BSJ), were used to transform maize (Hill=untransformed maize). The mg BChE/mg total soluble protein (tsp) based on activity relative to equine BChE (Sigma #1057) are given. We have identified high expressing seed, which corresponds to production of BChE at 50 mg/kg seed. FIGS. 2B and 2C show further analysis as percent total soluble protein, where data was collected for all seed produced, and also for the ten highest expressing seeds. In addition to the overall mean accumulation, a comparison of a selection of the highest expressing plants or seeds may actually be a better indication of the best potential expression for a given construct. As can be seen, the BSJ construct having the endosperm promoter and targeting to the vacuole expressed at a level of at least 0.04% for all seeds and at a level of at least 0.08% TSP when measuring the ten highest seed. The BSE construct having the endosperm promoter and cell wall targeting sequence produced BChE at levels of at least 0.09% TSP for all seed and at levels of at least 0.49% TSP when measuring the ten highest expressing seed (BSE). The BSK construct having the endosperm promoter and a cell wall signal sequence and endoplasmic reticulum targeting sequence expressed at levels of at least 0.20% TSP in all seed and at levels of at least 0.62% TSP when measuring the top ten seed. The constructs provide increasing expression of BChE at levels of at least 0.04, 0.5, 0.6, 0.7% TSP and more and amounts in-between.

BSE, BSK and BSJ are shown and have significant expression. The other constructs tested showed barely detectable or no expression. (Note here U/mg is units/mg and TSP refers to total soluble protein.) For all recombinant proteins we have tested to date, we have been able to increase accumulation of the product from 10 to 100-fold through selection over the course of a traditional backcrossing program into elite maize lines with optimal field characteristics. This is then followed by 1) “selfing” to create homozygous parents; 2) creating hybrid seed and; 3) growing the grain that can then be used to produce the protein (34). The earliest maize line produced, BSE, has been started in this program and has shown indications of increasing expression typical of other proteins we have produced. Based on this level of accumulation and our experience with more than 50 other recombinant proteins produced in maize grain, we estimate being able to achieve >500 mg BChE/kg grain after optimization based on these early lines that have already been identified.

In FIGS. 2B and 2C the letters above the graph reflect the result was statistically different. Statistical analysis was performed on two data sets. The first data set contained the top ten seeds in BChE activity for each construct. The second data set contained all seeds with positive BChE activity (higher than 0.0002 mg BChE per mg total soluble protein). For each data set, a nested, mixed model, analysis of variance (ANOVA) was performed. For these analyses, the natural logarithm of BChE was the response variable, and the factors were Construct, Event (nested within Construct), and Plant (nested within both Construct and Event). Construct was modeled as a fixed effect, while Event and Plant were modeled as random effects. Only the Construct factor was of interest since the goal was to assess differences in mean BChE level between the three constructs. (The Hi II negative control was tested in selected experiments from a frozen large-scale extraction and was not included in the statistical analysis). Significant differences between constructs were detected using Tukey's HSD procedure with α=0.05. All calculations were done in the SAS Institute's IMP software (version 11.1.1) using the restricted maximum likelihood (REML) algorithm. BuChE was analyzed on the logarithmic scale to correct for non-constant variance and non-normality of the data. As a result of this transformation, we compared the geometric means of BChE activity for each construct rather than the arithmetic means. Table 1 summarizes the number of events, plants, and seeds analyzed. The geometric mean expression of BChE as a percentage of total soluble protein (TSP) for each construct is shown for expressing constructs. Superscripts represent significant differences using Tukey's HSD test at α=0.05, with all positive seed (lower case) and top ten seeds (upper case) analyzed separately. bld=below limit of detection; nd=not done.

TABLE 1

| | | | | Mean % | Mean % |
|---|---|---|---|---|---|
| | Independent | | Positive | TSP all | TSP Top |
| Construct | Events | Plants | Seeds | Positive Seeds | Ten Seeds |
| BSA | 5 | 19 | bld | nd | nd |
| BSB | 3 | 9 | bld | nd | nd |
| BSC | 6 | 26 | bld | nd | nd |
| BSD | 16 | 32 | bld | nd | nd |
| BSE | 23 | 123 | 408 | 0.09^{b} | 0.49^{A} |
| BSF | 2 | 5 | bld | nd | nd |
| BSG | 11 | 21 | bld | nd | nd |
| BSJ | 14 | 54 | 64 | 0.04^{c} | 0.08^{B} |
| BSK | 8 | 62 | 220 | 0.20^{a} | 0.62^{A} |
| BSL | 8 | 30 | bld | nd | nd |

In general, purification of recombinant protein from maize grain is easier than most other systems as it has a low level of interfering phenolic compounds. In addition, high levels of endogenous protease inhibitors help preserve the protein as it is extracted. While the purification process for BChE from maize grain is still in development, an estimated cost can be compared to that for tobacco systems. In a recent review (35), it was estimated that tobacco-produced BChE could be produced at $1,210/dose but the hope was to bring this down further by increasing expression to 500 mg/kg with cost at $474/dose. This is a vast improvement over obtaining BChE from outdated blood. However, at this same level of expression in maize, production of the active ingredient would be <$1.00/gram. If the cost of purification is based on similar assumptions to those in the published tobacco model as well as on our own experience with other recombinant proteins produced form maize, we anticipate that the cost for the purified protein would be at least an order of magnitude less than the tobacco-produced version. Furthermore, as the protein is stable in grain for years, it is possible to simply store the BChE grain and perform just-in-time processing when the need arises.

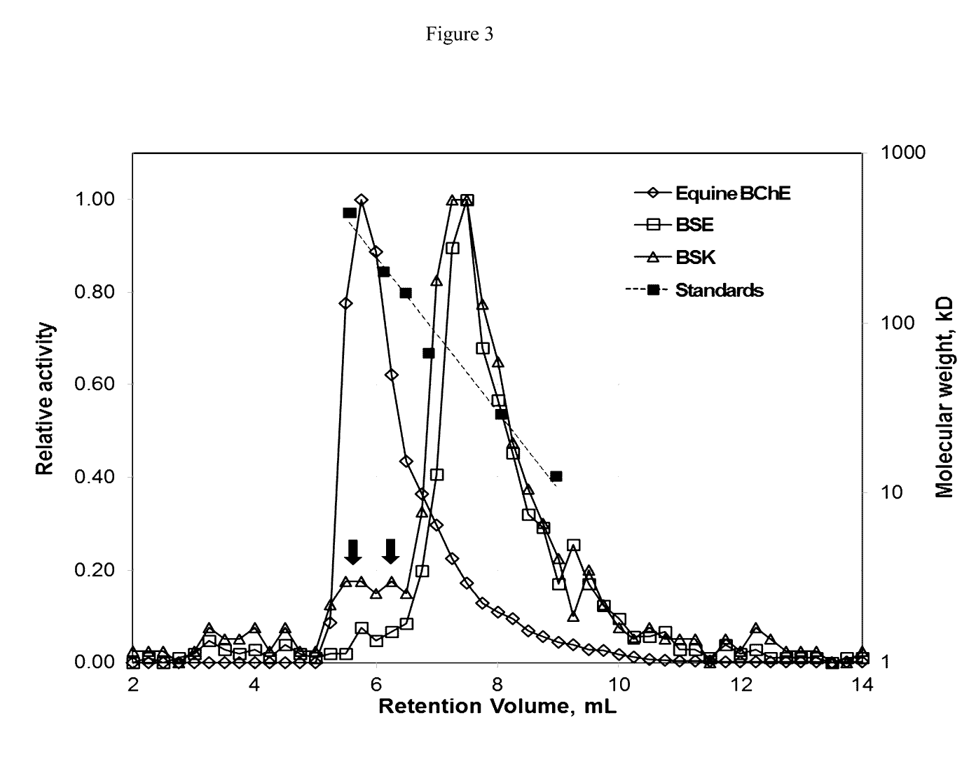

In addition to low cost, it is also desirable to produce the tetramer rather than the monomer in maize. This would reduce the amount of downstream processing/formulation of the material and help to maintain the overall low cost of production. For this reason, we examined the BChE from maize grain for its ability to make tetramers. It can be seen in FIG. 3 that the maize lines appear to make both tetramer and monomer, but the predominant form is the monomer. Extracts from pooled T₁ seed for constructs BSE and BSK were analyzed by size exclusion chromatography as described in Materials and Methods. A small increase in formation of higher molecular weight oligomers was observed with construct BSK (indicated by arrows). High molecular weight is at least about 340 kDa. Data points of the calibration regression are represented by closed symbols and dashed line (left axis), while relative activity of the three different BChE samples are represented as open symbols, solid lines (right axis). Dependence of formation of tetramers on subcellular localization was in general consistent with the Schneider et al. Schneider J D et al. (2014b) “Oligomerization status influences subcellular deposition and glycosylation of recombinant butyrylcholinesterase in Nicotiana benthamiana” Plant Biotechnology Journal 12:832-839

The overall goal of this work is to provide a low-cost, highly scalable production system for BChE. Maize offers one of the lowest costs of production with some of the fewest complications for purification, making it the system of choice for production of recombinant proteins (25-27). This assumes there is an adequate level of expression, and data to date demonstrates that BChE can be expressed in maize grain at levels that have the potential to enable a very low cost of production. Before optimizing this system, however, it would be greatly desirable to develop a maize line that predominantly produces the tetrameric rather than the monomeric form of the enzyme. As has been shown in other recombinant systems, it should be possible to favor formation of the tetramer by co-expressing a polyproline peptide (14). We will prepare two additional maize lines co-expressing polyproline peptides with the expectation that this will facilitate oligomerization of BChE. We will pursue further optimization of expression of the tetrameric form along with development of purification protocols. This should facilitate development of a relatively low-cost source of large amounts of tetrameric recombinant BChE.

Create Maize Lines with Increased Expression and Formation of Tetramers

The BSK construct provides evidence that tetramers can form on localization to the ER, but formation of a high proportion of tetrameric BChE is likely to require co-expression of a proline-rich polypeptide (polyprotein peptide). Therefore, a transcription unit with the tetramer-promoting polyproline peptide and a second transcription unit with the BuChE coding region will be prepared, both under control of the same promoter. Based on our initial data as to which tissue and intracellular compartment provide the highest levels of expression, we will utilize an endosperm-preferred promoter targeted to the ER. These constructs will be transformed into maize and T₁ plants will be analyzed for BuChE expression.

### Preparation of Two New Constructs and Agrobacterium Lines

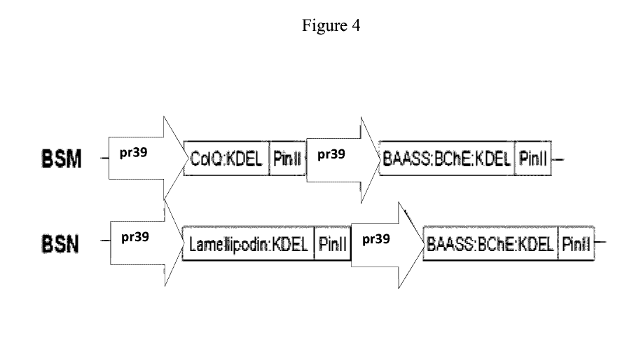

Two constructs will be prepared adding a polyproline peptide (PRAD) to constructs expressing BuChE in maize (FIG. 4). KDEL, (SEQ ID NO: 16), refers to the signal retaining expression in the endoplasmic reticulum. The first will incorporate the rQ45-PRAD modified rat collagen tail peptide sequence (gi:335892816) described in Duysen, et al. (14). As a variety of different polypeptides have been described as associated with BuChE tetramers, a second construct will be made with an alternative peptide, human lamellipodin (gi:82581557) (9). A synthesized 17 aa proline-rich peptide derived from a.a. 686-702 of human lamellipodin (abbreviated as hu28aa in the figure) was found to promote tetramerization but expression of the entire protein is likely to be necessary and further processing would occur inside the cell. It is not known exactly how the association between BChE and small polyproline peptides derived from lamellipodin occurs, although it has been proposed that after degradation in the cytosol by proteaseomes, proline-rich peptides are transported to the ER by proteins such as TAPs (transporters associated with antigen processing), allowing them to associate with nascent BChE (9). Thus, co-expression of two differed polyproline proteins to the ER will increase our chances of success.

The sequence of the two PRAD peptides will be optimized for maize codon usage and other features, such as mRNA destabilizing elements, which may impact expression. The resulting peptide coding sequences will be commercially synthesized (Blue Heron or GeneScript) with appropriate restriction sites for insertion into the pSB1/pSB11 vector system (36) for maize transformation under control of a maize endosperm-preferred promoter previously identified as supporting high levels of expression. Both peptides will be targeted to the ER. The potato protease inhibitor termination sequence (PinII) will be used. (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639). The well-established maize optimized phosphinothricin acetyltransferase (moPAT) gene driven by a cauliflower mosaic virus promoter will be used as a selectable marker for identification of transformed plants by herbicide screening. See, Gordon-Kamm et al., (1990) Plant Cell 2:603; Uchimiya et al., (1993) BioTechnology 11:835; White et al., Nucl. Acids Res. 18:1062, (1990); Spencer et al., 1990) Theor. Appl. Genet. 79:625-631, and Anzai et al., (1989) Mol. Gen. Gen. 219:492. A version of the PAT gene is the maize optimized PAT gene, described at U.S. Pat. No. 6,096,947.

The resulting vectors will be sequenced and analyzed by restriction digestion to confirm the absence of mutations or rearrangements. They will be transferred into the appropriate Agrobacterium strain EHA101 by standard tri-parental mating and electroporation procedures. KDEL (SEQ ID NO: 16) retains the expression in the endoplasmic reticulum, PinII refers to the PinII termination signal and pr39 refers to the pr39 promoter.

### Transformation of Maize and Producing T1 Generation Regenerated Plants and Screen for Expression

Embryos from maize line Hill at about 10 days after pollination will be mixed with the appropriate Agrobacterium strain harboring BChE coding sequence. As there can be significant variation in expression between independent transformation events, we will target production of at least 10 events for each construct. To generation regenerated plants will be moved from tissue culture to greenhouse and pollinated with maize line Hill to produce T₁ seed for analysis of expression. Six T₁ seed per plant will be analyzed for at least six plants from each of the events produced. For BChE this can be done by a modified version of the assay described by Ellman (37) that is already well-established in the lab. Briefly, individual seed are pulverized and extracted in Tris-saline buffer. Extracts are mixed with 5,5′-dithiobis(2-nitrobenzoate) (DNTB) in phosphate buffer to react with thiol groups in the sample, then mixed with butyrylthiocholine iodide and absorbance read at 412 nm in a 2 minute kinetic assay. For rQ45 the previously described modified protein included a FLAG tag sequence, presumably for detection of the protein. However, currently there are some options for commercial antibodies that may be usable with the N-terminal rat rQ45 protein. These include ab49190 from Abcam and ARP34362_p50 from Aviva Systems Biology, both raised to the N terminal amino acids 1-50 of human ColQ and predicted to cross-react with rat ColQ, and sc-69155 from Santa Cruz Biotechnology, also recognizing the N-terminus of ColQ. It should then be possible to use at least one of these antibodies to detect expression by western blot. The FLAG tag will therefore not be included and we expect to add a KDEL ER targeting sequence (SEQ ID NO: 16) in its place. For detection of the lamellipodin protein, it should also be possible to use commercially available antibodies to assess expression by western blot including sc-67603 and sc-68380 from Santa Cruz Biotechnology.

### Analysis of Oligomerization Status

The BuChE protein expressed in the constructs described above will be analyzed for quaternary structure. We will produce ˜1 kg of T₁ seed for each of our new constructs. This should provide sufficient material for preliminary studies on purification and oligomerization status.

### Purification

The purification process developed for Zea mays produced BChE (zm-BChE) is similar to that described for huBChE from outdated blood plasma or Cohn Fraction IV, which are typically combinations of affinity chromatography (procainamide-sepharose) and size-exclusion chromatography. Small batches are promising, but always problematic in terms of accurate assessment of yield. A typical current result is provided in Table 2, which demonstrates a 53-fold purification with an 11% yield. The activity of the zm-BChE, U/mg (U=μmole of butyrylthiocholine hydrolyzed per min) is ˜9 U/mg. While work remains to be done to achieve a higher level of purity and specific activity comparable to other systems, these results are comparable to those achieved with other enzymes such as OPH at a similar stage of development and show that the fundamental purification strategy is effective. Development and optimization of the process will continue as the availability of the raw material increases.

TABLE 2
Representative data of the zm-BChE purification process currently under
development.

| | | Total | | | |
|---|---|---|---|---|---|
| | Total | Protein, | Activity, | | Yield, |
| Sample | Units | mg | U/mg | Fold | % |
| Total Meal Extract | 600.9 | 3469.2 | 0.173 | 1 | 100 |
| AC-Con A | 126.9 | 28.5 | 4.45 | 26 | 21 |
| AC-Procanimide | 129.1 | 16.3 | 7.91 | 46 | 21 |
| SEC-G75 | 64.5 | 7.0 | 9.24 | 53 | 11 |

### Analysis of Oligomerization

Two activity based strategies will be used to assess oligomerization in the seed extract, and during purification. These include size exclusion chromatography and a gel electrophoresis approach based on that described by Karnovsky (Karnovsky, M. J. & Roots, L. (1964). A “direct-coloring” thiocholine method for cholinesterases. Journal of Histochemistry & Cytochemistry. 12: 219-221.

This project will result in maize lines with greater yields of the tetrameric form of BChE. The expression of two different polyproline peptides should increase our chances that at least one of the two will associate with BChE to allow formation of a high proportion of tetramers. Lines for high expression and analysis of oligomerization status in T₂ and subsequent generations will be investigated. This is expected to increase expression by at least 10-fold. With the appropriate collaborators, the exact combination of polyproline peptides that associate with BChE will be examined by Mass spectroscopy and Edmann degradation. Once expression of tetrameric BChE in quantities sufficient to achieve the target price (<$40/dose) is achieved, any additional improvements such as such as sialyl capping, will be addressed. This may include co-expression of additional genes allowing an appropriate glycosylation profile, or in-vitro processing of the plant-produced enzyme after isolation from seed. Studies of clearance time, in vitro binding of nerve agents and animal protection studies will be performed with the appropriate collaborators to demonstrate functional equivalency of the maize-produced BChE to human BCHE.

The foregoing is provided by way of illustration and is not intended to limit the scope of the invention. All references referred to are incorporated herein by reference.

### REFERENCES

1. Parikh K, et al. (2011) Gene-delivered butyrylcholinesterase is prophylactic against the toxicity of chemical warfare nerve agents and organophosphorus compounds. Journal of Pharmacology and Experimental Therapeutics 337(1):92-101.
2. Nachon F, Brazzolotto X, Trovaslet M, & Masson P (2013) Progress in the development of enzyme-based nerve agent bioscavengers. Chemico-biological interactions 206(3):536-544.
3. Ilyushin D, et al. (2013) Recombinant human butyrylcholinesterase as a new-age bioscavenger drug: development of the expression system. Acta naturae 5(1):73.
4. Masson P & Lockridge O (2010) Butyrylcholinesterase for protection from organophosphorus poisons: catalytic complexities and hysteretic behavior. Archives of biochemistry and biophysics 494(2): 107-120.
5. Blong R, BEDOWS E, & LOCKRIDGE O (1997) Tetramerization domain of human butyrylcholinesterase is at the C-terminus. Biochem. J 327:747-757.
6. Bon S, Ayon A, Leroy J, & Massoulié J (2003) Trimerization domain of the collagen tail of acetylcholinesterase. Neurochemical research 28(3-4): 523-535.
7. Lee H H, et al. (2004) Transcriptional Regulation of Acetylcholinesterase-associated Collagen ColQ DIFFERENTIAL EXPRESSION IN FAST AND SLOW TWITCH MUSCLE FIBERS IS DRIVEN BY DISTINCT PROMOTERS. Journal of Biological Chemistry 279(26):27098-27107.
8. Perrier A L, Massoulié J, & Krejci E (2002) PRiMA: the membrane anchor of acetylcholinesterase in the brain. Neuron 33(2):275-285.
9. Li H, Schopfer L, Masson P, & Lockridge O (2008) Lamellipodin proline rich peptides associated with native plasma butyrylcholinesterase tetramers. Biochem. J 411:425-432.
10. Biberoglu K, Schopfer L M, Tacal O, & Lockridge O (2012) The proline-rich tetramerization peptides in equine serum butyrylcholinesterase. FEBS Journal 279(20):3844-3858.
11. Lockridge O & Masson P (1999) Pesticides and susceptible populations: people with butyrylcholinesterase genetic variants may be at risk. Neurotoxicology 21(1-2):113-126.
12. Østergaard D, Viby-Mogensen J, Hanel H, & Skovgaard L (1988) Half-life of plasma cholinesterase. Acta anaesthesiologica scandinavica 32(3):266-269.
13. Saxena A, et al. (1998) Role of oligosaccharides in the pharmacokinetics of tissue-derived and genetically engineered cholinesterases. Molecular pharmacology 53(1):112-122.
14. Duysen E G, Bartels C F, & Lockridge O (2002) Wild-type and A328W mutant human butyrylcholinesterase tetramers expressed in Chinese hamster ovary cells have a 16-hour half-life in the circulation and protect mice from cocaine toxicity. Journal of Pharmacology and Experimental Therapeutics 302(2):751-758.
15. Magill A J (2012) DARPA Proposer's Day: Butyrylcholinesterase Expression in Plants.
16. Lockridge O, Schopfer L M, Winger G, & Woods J H (2005) Large scale purification of butyrylcholinesterase from human plasma suitable for injection into monkeys; a potential new therapeutic for protection against cocaine and nerve agent toxicity. The journal of medical, chemical, biological, and radiological defense 3:nihms5095.
17. Geyer B C, et al. (2010) Plant-derived human butyrylcholinesterase, but not an organophosphorous-compound hydrolyzing variant thereof, protects rodents against nerve agents. Proceedings of the National Academy of Sciences 107(47):20251-20256.
18. Huang Y-J, et al. (2007) Recombinant human butyrylcholinesterase from milk of transgenic animals to protect against organophosphate poisoning. Proceedings of the National Academy of Sciences 104(34):13603-13608.
19. Geyer B C, et al. (2010) Transgenic plants as a source for the bioscavenging enzyme, human butyrylcholinesterase. Plant biotechnology journal 8(8): 873-886.
20. Larrimore K E, et al. (2013) Plants as a source of butyrylcholinesterase variants designed for enhanced cocaine hydrolase activity. Chemico-biological interactions 203(1):217-220.
21. Schneider J D, et al. (2014) Expression of human butyrylcholinesterase with an engineered glycosylation profile resembling the plasma-derived orthologue. Biotechnology journal 9(4):501-510.
22. Schneider J D, et al. (2014) Oligomerization status influences subcellular deposition and glycosylation of recombinant butyrylcholinesterase in Nicotiana benthamiana. Plant biotechnology journal.
23. Baldassarre H, et al. (2008) Lactation performance of transgenic goats expressing recombinant human butyryl-cholinesterase in the milk. Transgenic research 17(1):73-84.
24. Rosenberg Y (2013) Pretreatment or post exposure treatment for exposure to a toxic substance by pulmonary delivery (inhaler) of a bioscavenger. (Google Patents).
25. Howard J & Hood E E (2014) The Future of Plant-Produced Pharmaceuticals and Industrial Proteins. Commercial Plant-Produced Recombinant Protein Products, (Springer), pp 261-274.
26. Howard J A & Hood E (2005) Bioindustrial and biopharmaceutical products produced in plants. Advances in Agronomy, Vol 85 85:91-124.
27. Howard J A & Hood E E (2007) Methods for Growing Nonfood Products in Transgenic Plants. Crop science 47(3): 1255-1262.
28. Howard J A & Hood E (2005) Bioindustrial and biopharmaceutical products produced in plants. Advances in agronomy 85:91-124.
29. Hood E E, et al. (1997) Commercial production of avidin from transgenic maize: characterization of transformant, production, processing, extraction and purification. Molecular Breeding 3(4):291-306.
30. Hood E E, et al. (2007) Subcellular targeting is a key condition for high-level accumulation of cellulase protein in transgenic maize seed. Plant Biotechnol J 5(6):709-719.
31. Egelkrout E, et al. (2013) Enhanced Expression Levels of Cellulase Enzymes Using Multiple Transcription Units. BioEnergy Research 6(2):699-710.
32. Woodard S, et al. (2003) Maize (Zea mays)-derived bovine trypsin: characterization of the first large-scale, commercial protein product from transgenic plants. Biotechnology and applied biochemistry 38(2):123-130.
33. Hood E E, et al. (2007) Subcellular targeting is a key condition for high-level accumulation of cellulase protein in transgenic maize seed. Plant biotechnology journal (6):709-719.
34. Hood E E, et al. (2012) Manipulating corn germplasm to increase recombinant protein accumulation. Plant Biotechnology Journal 10(1):20-30.
35. Tusé D, Tu T, & McDonald K A (2014) Manufacturing Economics of Plant-Made Biologics: Case Studies in Therapeutic and Industrial Enzymes. BioMed Research International 2014.
36. Ishida Y, et al. (1996) High efficiency transformation of maize (Zea mays L.) mediated by Agrobacterium tumefaciens. Nat Biotechnol 14(6):745-750.
37. Ellman G L, Courtney K D, & Featherstone R M (1961) A new and rapid colorimetric determination of acetylcholinesterase activity. Biochemical pharmacology 7(2):88-95.

What is claimed is:

1. A method of increasing expression of Butyrylcholinesterase (BChE) in a plant, plant part or plant cell, comprising introducing into said plant, plant part or plant cell a construct comprising a promoter preferentially expressing to endosperm cells of the plant operably linked to a nucleic acid molecule encoding BChE.

2. The method of claim 1, said construct further comprising a nucleic acid molecule that targets expression to the cell wall.

3. The method of claim 1, said construct further comprising a nucleic acid molecule that targets expression to the endoplasmic reticulum.

4. The method of claim 3, said construct further comprising a nucleic acid molecule that targets expression to the cell wall.

5. The method of claim 4, wherein said expression of said BChE is at least 0.5% TSP.

6. The method of claim 4, wherein expression of said BChE is at a level of at least 50 mg/kg of plant seed.

7. The method of claim 2 wherein said nucleic acid molecule that targets expression to the cell wall comprises SEQ ID NO: 6.

8. The method of claim 3, wherein said nucleic acid molecule that targets expression to the endoplasmic reticulum encodes SEQ ID NO: 16 or 17.

9. The method of claim 4, wherein said nucleic acid molecule that targets expression to the cell wall comprises SEQ ID NO: 6 and the nucleic acid molecule that targets expression to the endoplasmic reticulum comprises a molecule encoding SEQ ID NO: 16 or 17.

10. The method of claim 1, wherein said plant, plant part or plant cell is a maize plant, plant part or plant cell.

11. A method of increasing expression of Butyrylcholinesterase (BChE) in a plant, plant part or plant cell, the method comprising introducing into said plant, plant part or plant cell a construct comprising a nucleic acid molecule encoding BChE operably linked to a promoter preferentially expressing to endosperm cells of the plant, plant part or plant cell and a nucleic acid molecule targeting expression to the cell wall, and measuring the amount of BChE expressed, wherein said expression is higher than expression of a construct comprising a nucleic acid molecule encoding BChE operably linked to a promoter not preferentially expressing to endosperm cells and which does not comprise a nucleic acid molecule targeting expression to the cell wall.

12. The method of claim 11, said construct further comprising a nucleic acid molecule that targets expression to the endoplasmic reticulum.

13. A maize plant, plant part or plant cell having increased expression of Butyrylcholinesterase (BChE) said plant comprising a construct comprising a promoter preferentially expressing to endosperm cells of the plant operably linked to a nucleic acid molecule encoding BChE.

14. The plant of claim 13, said construct further comprising a nucleic acid molecule that targets expression to the cell wall.

15. The plant of claim 13, said construct further comprising a nucleic acid molecule that targets expression to the endoplasmic reticulum.

16. The plant of claim 15, said construct further comprising a nucleic acid molecule that targets expression to the cell wall.

17. The plant of claim 15, wherein said expression of said BChE is at least 0.5% TSP.

18. The plant of claim 15, wherein expression of said BChE is at a level of at least 50 mg/kg of plant seed.
